(19) United States
(12) Patent Application Publication
(10) Pub. No.: US 2020/0221763 A1
(43) Pub. Date: Jul. 16, 2020

(54) ELECTRONIC CIGARETTE

(71) Applicant: Healthier Choices Management Corp, Hollywood, FL (US)

(72) Inventors: Gilbert Cyphert, Phoenix, AZ (US); Edwin Balder, Mesa, AZ (US); Daniel Julia, Phoenix, AZ (US); Jeffrey E. Holman, Hollywood, FL (US)

(21) Appl. No.: 16/837,761

(22) Filed: Apr. 1, 2020

Related U.S. Application Data
(63) Continuation of application No. 15/796,618, filed on Oct. 27, 2017.
Application No. 15/796,618 is a continuation-in-part of application No. 15/786,364, filed on Oct. 17, 2017, now abandoned.
Application No. 15/786,364 is a continuation-in-part of application No. 15/298,147, filed on Oct. 19, 2016, now abandoned.
Application No. 15/298,147 is a continuation of application No. 13/999,652, filed on Mar. 14, 2014, now Pat. No. 9,538,788.
(60) Provisional application No. 61/852,336, filed on Mar. 15, 2013.

(51) Int. Cl. A24D 3/04; A61M 15/06; A24F 7/04; A24D 3/10; A61M 11/00; A61M 11/04

(52) U.S. Cl. CPC A24D 3/048; A61M 15/06; A24F 7/04; A24D 3/10; A61M 2205/3653; A61M 11/042; A61M 2205/75; A61M 2205/8206; A61M 11/003

(57) ABSTRACT

A heating device for an electronic smoking apparatus has a heating element encased in a bulb so that e-liquid does not come in contact with the heating element, but rather is heated indirectly by the bulb. The heating element is in electrical communication with electrical contacts exterior of the bulb, which contacts are placed in electrical communication with a power source such as a batter. The bulb can be made of glass, quartz, or similar inert silica-based compositions. The bulb can be shaped and sized to accommodate the components of a variety of electronic smoking apparatus. The bulb includes an indentation or an external protrusion that functions as a reservoir to receive and retain inhalant material such as liquids, concentrates or herbal material.

### CROSS REFERENCE TO RELATED APPLICATIONS

This application is a continuation of U.S. application Ser. No. 16/796,618 filed Oct. 27, 2017, which is a continuation-in-part of U.S. application Ser. No. 15/786,364 filed Oct. 17, 2017, which is a continuation-in-part of 15/298,147 filed Oct. 19, 2016, which is a continuation of U.S. application Ser. No. 13/999,652 filed Mar. 14, 2014, now U.S. Pat. No. 9,538,788, which claims priority to U.S. Provisional Application No. Ser. No. 61/852,336, filed Mar. 15, 2013

### BRIEF DESCRIPTION OF THE DRAWINGS

FIG. 1 is a cross-sectional view of one embodiment of an electronic cigarette embodying the principals of the invention;

FIG. 2 is a cross-sectional view of a second embodiment of an electronic cigarette embodying the principals of the invention;

FIG. 3 is a cross-sectional view of a portion of the second embodiment of an electronic cigarette illustrated in FIG. 2;

FIG. 3A is a perspective view of an embodiment of a heating device.

FIG. 3B is a perspective view of another embodiment of a heating device.

FIG. 3C is a perspective view of yet another embodiment of a heating device.

FIG. 3D is a perspective view of yet another embodiment of a heating device.

FIG. 3E is a perspective view of an embodiment of a bulb of the heating device.

FIG. 3F is a perspective view of another embodiment of a bulb of a heating device.

FIG. 3G is a perspective view of yet another embodiment of a bulb of a heating device.

FIG. 3H is a perspective view of yet another embodiment of a bulb of a heating device.

FIG. 3I is a perspective view of yet another embodiment of a bulb of a heating device.

FIG. 3J is a perspective view of yet another embodiment of a bulb of a heating device.

FIG. 4 is a view of an electronic cigarette showing a moveable clip feature;

FIG. 5 is a view of an electronic cigarette showing a retractable tip feature;

FIG. 6 is a view of an electronic cigarette showing a peel-away wrapper tip;

FIG. 7 is a view of the peel-away wrapper illustrated in FIG. 6;

FIG. 8 is an end view of a filter element that may be included in a embodiment of an electronic cigarette; and

FIG. 9 is a view of a removable sleeve that may be attachable to an electronic cigarette

FIG. 10 is a perspective view of the bulb heater in use.

It will be recognized that some or all of the Figures are schematic representations for purposes of illustration and do not necessarily depict the actual relative sizes or locations of the elements shown. The Figures are provided for the purpose of illustrating one or more embodiments of the invention with the explicit understanding that they will not be used to limit the scope or the meaning of the claims.

### DETAILED DESCRIPTION OF THE INVENTION

In the following description, for the purposes of explanation, numerous specific details are set forth in order to provide a thorough understanding of the electronic cigarette of the present invention. It will be apparent, however, to one skilled in the art that the electronic cigarette may be practiced without some of these specific details. Throughout this description, the embodiments and examples shown should be considered as exemplars, rather than as limitations on the electronic cigarette. That is, the following description provides examples, and the accompanying drawings show various examples for the purposes of illustration. However, these examples should not be construed in a limiting sense as they are merely intended to provide examples of the electronic cigarette rather than to provide an exhaustive list of all possible implementations of the electronic cigarette.

Specific embodiments of the invention will now be further described by the following, non-limiting examples which will serve to illustrate various features. The examples are intended merely to facilitate an understanding of ways in which the invention may be practiced and to further enable those of skill in the art to practice the invention. Accordingly, the examples should not be construed as limiting the scope of the invention. In addition, reference throughout this specification to “one embodiment” or “an embodiment” means that a particular feature, structure or characteristic described in connection with the embodiment is included in at least one embodiment of the present invention. Thus, appearances of the phrases “in one embodiment” or “in an embodiment” in various places throughout this specification are not necessarily all referring to the same embodiment. Furthermore, the particular features, structures or characteristics may be combined in any suitable manner in one or more embodiments.

### Electronic Cigarette Construction

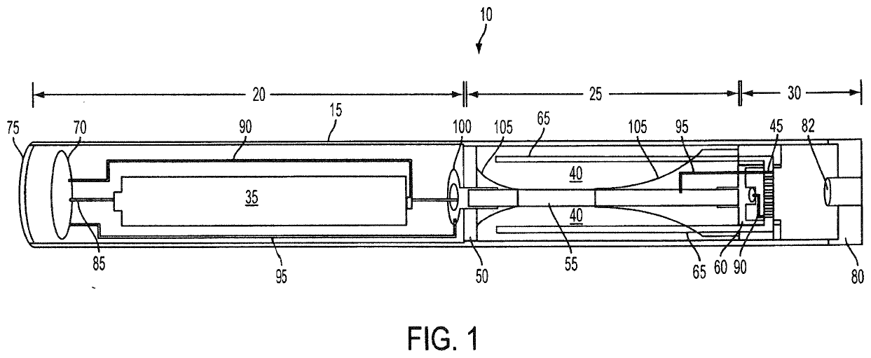

Referring to FIG. 1, an electronic cigarette 10 is illustrated. As shown in FIG. 1, the electronic cigarette 10 comprises a cylindrical casing, or tube 15 that may be constructed of stainless steel, plastic, or other materials. Within the cylindrical casing 15, a first chamber 20, a second chamber 25, and a third chamber 30 are formed, the three chambers located sequentially within the cylindrical casing 15.

A battery 35 is located in the first chamber 20, a inhalant material such as a liquid or concentrate 40 is located in the second chamber 25, and a heating element 45 is located in the third chamber 30. In one embodiment, the liquid 40 contained in the electronic cigarette 10 comprises: glycol, nicotine, water based flavors of regular or menthol (or other desired flavors). The heating element 45 may be made of platinum wire, nickel chromium alloy or iron chromium aluminum alloy wire with a rare earth element. The battery 35 may be lithium, but other materials and types of batteries may be employed, including disposable types.

A first seal 50 forms a partition between the first 20 and second 25 chambers. The first seal 50 may be constructed of rubber, silicone, plastic or other materials. The first seal includes a hole or aperture in which a portion of a tube 55 is located. The tube 55 extends substantially the length of the second chamber 25 allowing passage of air from the first chamber 20 into the third chamber 30.

A second seal 60 forms a partition between the second 25 and third 30 chambers. In one embodiment, the second seal 60 comprises a ceramic material shaped substantially in a “T.” The “leg” of the “T” comprises a hollow cylinder that receives one end of the tube 55 and the “top” of the “T” comprises two arms that form the seal between the second 25 and third 30 chambers, and may also hold, or fix the heating element 45 in position in the third chamber 30.

The electronic cigarette 10 also includes a cotton or other fiber material 65 that has a first segment in contact with the heating element 45, and a second segment located in the second chamber 25, so that the fiber material 65 can carry or transfer the fluid 40 from the second chamber 25 to the heating element 45.

In one embodiment, the fiber material comprises a high-temperature fiberglass fabric, but other materials may be employed, such as cotton, high-temperature conduction oil cotton, or other materials. The fiber material 65 also may be positioned by the second seal 60 to aid in sealing the second chamber 25 from the third chamber 30.

An air pressure switch is located at a distal end of the first chamber 20 and may be mounted on, or separate from a printed circuit board 70. A light-emitting diode 75 (LED) is located adjacent to the printed circuit board 70, with the LED 75 forming an end-cap at one end of the electronic cigarette 10. A mouthpiece (not shown) may be located at the other end of the electronic cigarette 10, with the mouthpiece coupled to the end-cap 80 that includes a small aperture, or end-cap aperture 82 that allows passage of air from the third chamber 30 though the mouthpiece and into a user's mouth. The end-cap 80 may be made of silicone, plastic, metal or other materials.

The printed circuit board, or chip 70 includes the air pressure switch, which may be integral to the chip or may be a separate element that communicates with the chip. In one embodiment, the chip and air pressure switch comprise an electronic assembly that allows communication between the air pressure switch and the other components comprising the printed circuit board. For example, in one embodiment, the printed circuit board, or chip may comprise an embedded data processor connected via an internal bus to a read only memory containing the executable code for causing the microprocessor o perform the functions described herein. In another embodiment, the printed circuit board, or chip may comprise one or more electronic circuits that employ one or more switches to perform the functions described herein.

As shown in FIG. 1, the printed circuit board, or chip 70 has three wires coupled to it, the anode wire 85, the cathode wire 90 and the air pressure switch wire 95. The anode wire 85 runs from the printed circuit board 70 to the anode side of the battery. The cathode wire 90 connects the printed circuit board 70 to the cathode side of the battery, and then passes through the first seal 50 and into tube 55 connecting to a first end of the heating element 45 by extending all the way through the tube 55. The air pressure switch wire 95 connects the air pressure switch to the second end of the heating element 45, by also passing through the first seal 50 and then into the tube 55.

Sealing member 100 may be coupled to the tube 55 to provide additional sealing to prevent passage of fluid 40 from the second compartment 25 into the first compartment 20. The sealing member 100 may comprise a cylindrical shape at one end that receives the tube 55 and a shoulder or flange at the other end that aids in sealing the hole or aperture in the first seal 50 seal.

In addition, two sealing tubes 105 are included to seal each end of the second chamber 25. As shown in FIG. 1, one sealing tube 105 contacts the first seal 50, with the other end contacting the entire perimeter, or circumference of tube 55 so that the area where the tube 55 engages with the first seal 50 is kept free of the fluid 40. Similarly, at the other end of the second chamber 25, a second sealing tube 105 contacts the entire perimeter of tube 55 with a distal end enclosing the elements located at the end of the second chamber 25. However, the fiber material 65 extend through holes in the sealing tube 105 so that fluid 40 can pass along the fiber material 65 to the heating element 45. But, the air pressure switch wire 95, and cathode wire 90, as well as the end of the tube 55 that engages with the second seal 60 are kept free of the fluid 40. Optionally, cotton batting (not shown) may be positioned around the tube 55 and under, or within the two sealing tubes 105 to aid in sealing so that the fluid 40 does not reach either end of the tube 55, or the other areas sealed by the two sealing tubes 105.

An air inlet (not shown), in the form of a hole or aperture, may be located in the external wall of the casing 15 allowing passage of ambient air into the first chamber 20.

The printed circuit board, or chip 70 in conjunction with the air pressure switch controls the function of the electronic cigarette 10. According to one embodiment, outside, or ambient air is drawn into the first chamber 20 through the air inlet (not shown). The air pressure switch senses the low pressure formed by a user “drawing” on the mouthpiece (not shown), and activates the heating element 45 by allowing electricity to flow from the battery to the heating element 45. The LED 75 also illuminates at this time. The air flows down the tube 55 and into the third chamber 30, where the heating element 45 has atomized, or vaporized some of the fluid 40 that is carried to the heating element 45 on the fiber material 65. The vaporized fluid mixes with the air, and passes though end-cap aperture 82 that allows passage of the mixture from the third chamber 30 though the mouthpiece and into a user's mouth.

In one embodiment, the electronic cigarette 10 is disposable, During operation, if a user smokes for more than 5 seconds per puff the LED flashes two times then shuts off, or will flash ten times, which indicates the battery 35 is running low on stored power.

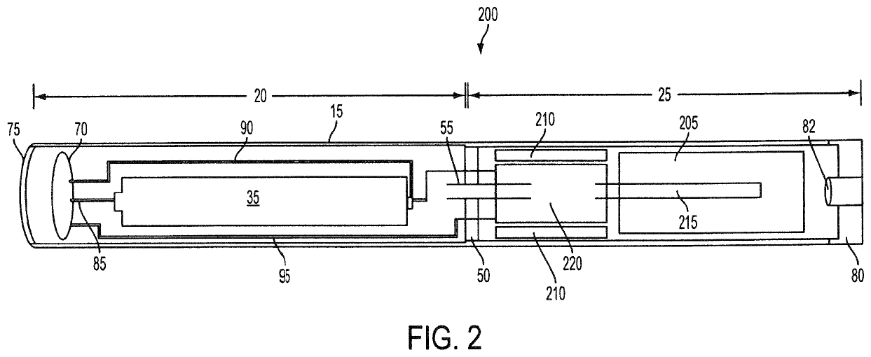

Referring to FIG. 2, another embodiment electronic cigarette 200 is illustrated. As shown in FIG. 2, the electronic cigarette 200 comprises a cylindrical casing, or tube 15 that may be constructed of stainless steel, plastic, or other materials. Within the cylindrical casing 15, is a first chamber 20 and a second chamber 25 are formed, the two chambers located within the cylindrical casing 15.

A battery 35 is located in the first chamber 20, and a fluid container 205 is located in the second chamber. The fluid container 205 contains a liquid mixture such as glycol, nicotine, water based flavors of regular or menthol (or other desired flavors). The battery 35 may be lithium, but other materials and types of batteries may be employed, including disposable types, such as AAA or AA sized batteries.

A first seal 50 forms a partition between the first 20 and second 25 chambers. The first seal 50 may be constructed of rubber, silicone, plastic or other materials. The first seal includes a hole or aperture in which a portion of a tube 55 is located. The tube 55 extends into a heating assembly 220 and allows passage of air from the first chamber 20 into the second chamber 25.

Electronic Cigarette with Bulb-Encased Heating Device

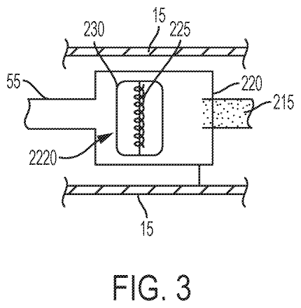

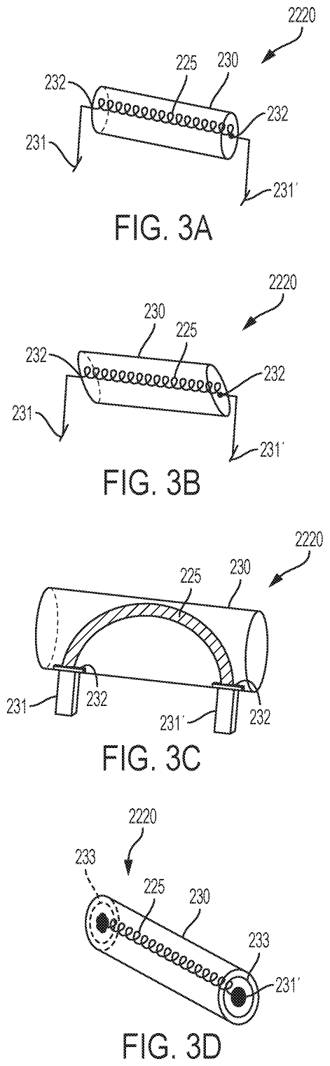

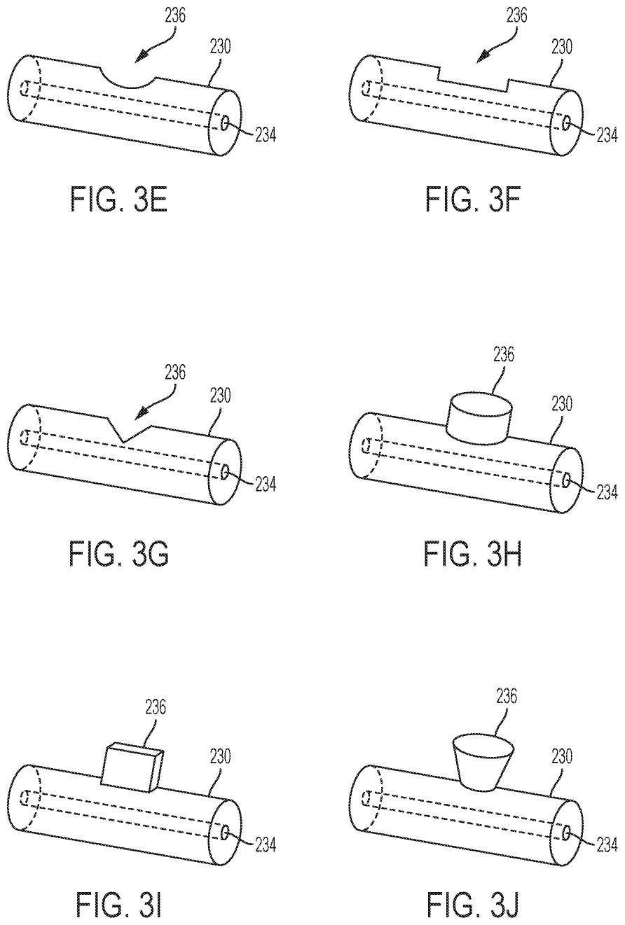

Referring now to FIG. 3, in one embodiment, the tube 55 comprises a ceramic, brass, or other metal material shaped substantially in a “T.” The “leg” of the “T” comprises a hollow cylinder that comprises one end of the tube 55 and the “top” of the “T” comprises two arms that hold, or fix a heating device 2220 in position in the heating assembly 220. In some embodiments, the heating device 2220 comprises a heating element 225 encased inside a bulb 230. The heating element 225 receives electrical energy (current) from the battery which causes the element 225 to heat up, which in turns heats the bulb 230, which bulb 230 then heats the fluid that is introduced into the heating assembly 220 through fiber element 215 that connects the heating assembly 220 with the fluid container 205. One or more apertures (not shown) allow the passage of the vapor that is produced when the fluid contacts the heated glass bulb 230. In some embodiments, the heating element 225 is be made of platinum wire, nickel chromium alloy or iron chromium aluminum alloy wire with a rare earth element. The tempered glass bulb 230 may also be manufactured from quartz, or other elements such as those described further herein. One feature of this arrangement is that the fluid is not brought into direct contact with the heating element 225 but rather contacts the heated bulb 230, avoiding any chemical reaction between the fluid and the heating element that would otherwise release harmful toxins such as heavy metals into the inhaled vapor.

In one embodiment, the fiber element 215 comprises a high-temperature fiberglass fabric, but other materials may be employed, such as cotton, high-temperature conduction oil cotton, or other materials. Sealing material 210 located around the heating assembly 220 prevents any fluid from passing into the first chamber 20, and may also be constructed of a high-temperature fiberglass fabric, but other materials may be employed, such as cotton, high-temperature conduction oil cotton, or other materials. The vapor then passes around the fluid container 205 and out through the end-cap aperture 82.

An air pressure switch is located at a distal end of the first chamber 20 and may be mounted on, or separate from a printed circuit board 70. A light-emitting diode 75 (LED) is located adjacent to the printed circuit board 70, with the LED 75 forming an end-cap at one end of the electronic cigarette 200. A mouthpiece (not shown) may be located at the other end of the electronic cigarette 200, with the mouthpiece coupled to the end-cap 80 that includes a small aperture, or end-cap aperture 82 that allows passage of air from the second chamber 25 though the mouthpiece and into a user's mouth. The end-cap 80 may be made of silicone, plastic, metal or other materials.

The printed circuit board, or chip 70 in conjunction with the air pressure switch controls the function of the electronic cigarette 200. According to one embodiment, outside, or ambient air is drawn into the first chamber 20 through the air inlet (not shown). The air pressure switch senses the low pressure formed by a user “drawing” on the mouthpiece (not shown), and activates the heating element 225 by allowing electricity to flow from the battery to the heating element 225. The LED 75 also illuminates at this time. The air flows down the tube 55 and into the heating assembly 220, where the glass bulb 230 has atomized, or vaporized some of the fluid that is carried to the heating assembly 220 by the fiber element 215. The vaporized fluid mixes with the air, and passes though end-cap aperture 82.

As shown in FIG. 2, the printed circuit board, or chip 70 has three wires coupled to it, the anode wire 85, the cathode wire 90 and the air pressure switch wire 95. The anode wire 85 runs from the printed circuit board 70 to the anode side of the battery. The cathode wire 90 connects the printed circuit board 70 to the cathode side of the battery, and then passes through the first seal 50 and into tube 55 connecting to the heating assembly 220 by extending all the way through the tube 55. The air pressure switch wire 95 connects the air pressure switch to the heating assembly 220, by also passing through the first seal 50.

### Universal Bulb-Encased Heating Device

Referring to FIGS. 3A-3D, shown are various non-exhaustive embodiments of a universal bulb-encased heating device 2220 that can be configured for use as part of the heating assembly 220 of an electronic smoking device such as electronic cigarette 200 or can be applied to a multi-component modular electronic smoking device or vaporizer device such as, without limitation, a two-piece tank-battery type vaporizer, a re-buildable dripping atomizer (RDA) or a re-buildable dripping tank atomizer. In some cases these modular electronic smoking devices are called vaporizer “mods,” i.e. module and/or modified, wherein the user configures the components of the vaporizer to perform in a desired way, often with respect to the volume, intensity, density, and flavor of the vapor. The heating device 2220 comprises an heating element 225 disposed in and encased by a bulb 230. In some embodiments the bulb comprises quartz, glass, silica-containing compositions, or any like composition that is capable of protecting and containing the heating element 225 from exposure during use. In some embodiments, the bulb 230 is substantially cylindrical as shown in FIG. 3A and may also be configured with beveled ends as shown in FIG. 3B. In some embodiments, as shown in FIGS. 3E-3J, the heating element 225 is received in through-bore 234 of the bulb 230 for a secure, retained interference fit. The heating element 225 may optionally be glued or sealed to the bulb 230.

The heating element 225 comprises a heat conductive wire or coil as shown in FIGS. 3A and 3B. In other embodiments, the heating element 225 comprises a heat-conductive ribbon as shown in FIG. 3C. In some embodiments, the ends of the heating element 225 are in electrical communication exterior of the bulb 230 to electrical contacts 231 and 231′. The electrical contacts 231 and 231′ define the respective anode and cathode of the electrical circuit between the heating element 225 and the power source of the device, such as battery 35. In some embodiments, the heating element 225 itself extends exterior of the bulb and terminates at the contacts 231 and 231′. In other embodiments, the heating element 225 is sealed within the bulb 230 with discrete electrical contacts 231 and 231′ structured to be in electrical communication with the heating element 225.

In some embodiments, such as in FIGS. 3A and 3B, the electrical contacts 231 and 231′ are configured to be secured to the power posts of an RDA or RDTA or other like vaporizer power supply hardware by a fastener such as a screw. In other embodiments, such as in FIG. 3C, the contacts 231 and 231′ are received in a slot or otherwise achieve and interference fit with a power source or power adaptation point.

In yet another embodiment, as shown in FIG, 3D the contacts 231 and 231′ are flush with the exterior surface of the bulb 230, providing surface-type flat electrical contacts. The flat contacts can be coupled with a magnetic connector element 233 surrounding or embedded therein to provide a releasable magnetic feature that connects to a target power source or related hardware. Accordingly, the configuration of the heating element 225 and the bulb 230 can be varied depending on the application and whether the heating device 2200 is intended to be permanently affixed as part of the electronic smoking apparatus, or whether it is designed to be modular, removable and replaceable.

In some embodiments, the heating element 225 is sealed to the respective ends of the bulb 230 by a sealing member 232. The sealing member 232 comprises a heat resistant polymer sealant, plug, grommet, or washer type element that is capable of sealing the ends of the bulb 230 at the insertion point of the heating element 225. In other embodiments the bulb 230 may be itself crimped or heat sealed to the heating element 225 at the ends thereof. Yet still, the heating element 225 may be itself glued into through-bore 234 (See FIGS. 3E-3J) using a heat resistant polymer sealant or other epoxy or adhesive-like sealant material.

In FIG. 3C, shown is another embodiment of the heating device 2220 configured with a ribbon-type heating element 220. The bulb 230 encases the ribbon, which ribbon provides larger surface area than a the wire or coil-type heating element shown in FIGS. 3A and 3B. FIG. 3C also demonstrates another embodiment of the contacts 231 and 231′ here comprising elongated tabs that are configured to be received by power supply slots on a target RDA or RTDA or other structure of an electronic cigarettes 200 or vaporizer device.

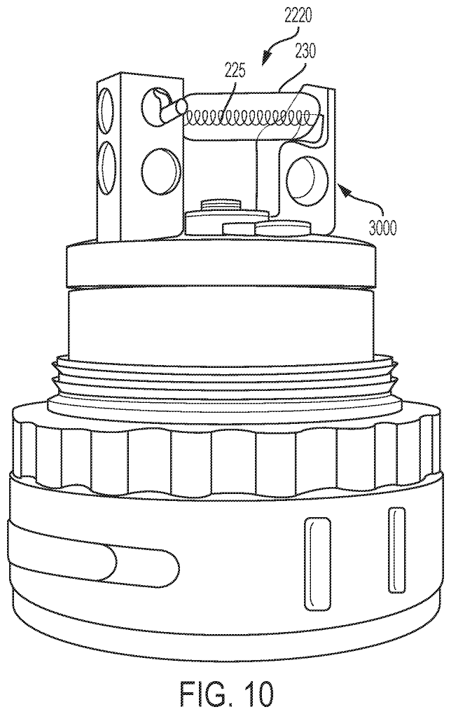

It is appreciated that the bulb 230 may be comprise various shapes and sizes in order to be compatible with the given application such as in disposable “closed system” electronic cigarettes, modular vaporizer units and RDA/RDTA units. For example, FIG. 10 depicts an embodiment of the heating device 2200 configured with beveled ends that conform to the posts of an exemplary RDTA 3000. Here, the contacts 231 and 231′ are physically attached to and electrically coupled. to the bi-lateral posts 3001 of the RDA 3000 to provide a secure connection. The heating device 2200 is configured to be in fluid communication with a vaporization fluid such as, for example, by a fiber element, wick, capillary-action tube or other means that can bring such fluid into contact with the surface of the bulb 230. In some embodiments, the shape of the bulb 230 can be manipulated to change the available exposed surface area.

With the heating device 2220 in electrical communication with a power source (such as a battery 35 or other external or internal battery source), the heating element 225 will heat up and, in turn, heat the bulb 230. Once adequate temperature levels are reach, inhalation (smoking) material such as fluid (e-liquid), concentrates (waxes), dry material (herbal material, tobacco) coming in contact with the surface of the bulb will vaporize into an vapor or like material suitable for inhalation.

It is appreciated and understood that the bulb-type heating device 2220 provides a substantial health benefit over traditional wire/wick designs in that the bulb encases the heating element, thereby preventing a potentially toxic reaction between the target vaporization fluid and the heating element. In this design, the heating device 2200 is designed such that the fluid will only come into physical contact with the inert bulb (glass, quartz, or the like), which bulb is heated by the internal heating element 220. Thus, the fluid is indirectly heated by the heating element 225, which element 225 generates heat through the application of electrical current therethrough, from a power supply and connection means of any known type. The avoidance of direct contact between the fluid and the heating element 220 also has the further advantage of improving flavor and vapor concentration as it provides a cleaner vaporization reaction. To wit, empirical testing has shown that use of the bulb-encased heating device 2220 reduces the presence of heavy metal and other toxins in the vapor by substantial levels over traditional exposed coil-type heating means. In some non-limiting exemplar studies, up to 95% of heavy-metal toxins were reduced.

Bulb-Encased Heating Device with Reservoir Feature

With reference to FIGS. 3E-3J, in some embodiments the bulb 230 can be fashioned with one or more reservoirs 236 disposed thereon, the reservoir configured to receive and retain inhalation (smoking) material such as fluid (e-liquid), concentrates (waxes), dry material (herbal material, tobacco) in order to eliminate any complex wicking systems and put the inhalation material directly in contact with the blub 230 in a secure manner. In some embodiments, such as shown in FIGS. 3E, 3F, and 3G, the reservoir 236 comprises one or more indentations or impressions on the surface of the bulb 230, thus providing an integrated reservoir to retain the inhalation material. The reservoir 236 configured as an indentation may comprise various shapes such as circular, cylindrical, elliptical, oval, conical, square, rectangular, and the like. FIG. 3E shows the reservoir as a circular or oval indentation, FIG. 3F shows the reservoir 236 as a square or rectangular indentation, and FIG. 3G shows the reservoir 236 as a conical or V-shaped indentation.

With reference to FIGS. 3H-3J, in some embodiments the reservoir 236 comprises a discrete protrusion extending from the bulb that is either removably attached to or integrated with the bulb 230 to receive and retain smoking material such as a fluids and concentrates. As demonstrated in the exemplary FIGS. 3H, 3I, and 3J, the externally-configured reservoir 236 may comprise various shapes such as circular, cylindrical, elliptical, oval, conical, square, rectangular, and the like. FIG. 3H shows the reservoir 236 as a cylindrical protrusion, and FIG. 3I shows the reservoir 236 as a square or rectangular protrusion, and FIG. 3J shows the reservoir 236 as a conical protrusion.

Further, as mentioned above and as shown in FIGS. 3E-3J, in some embodiments the bulb 230 includes a through-bore 234 disposed lengthwise through the bulb 230 that is configured to received and retain a heating element 225. The through-bore 234 provides added convenience to replace the heating element 225 as desired for different applications or if the heating element 225 were to wear out. The through-bore 234 is also particularly useful in the case that bulbs 230 are sold separately as the user can select a heating element 225 to fit the user's desired performance and specifications.

### Extendable Tip Feature

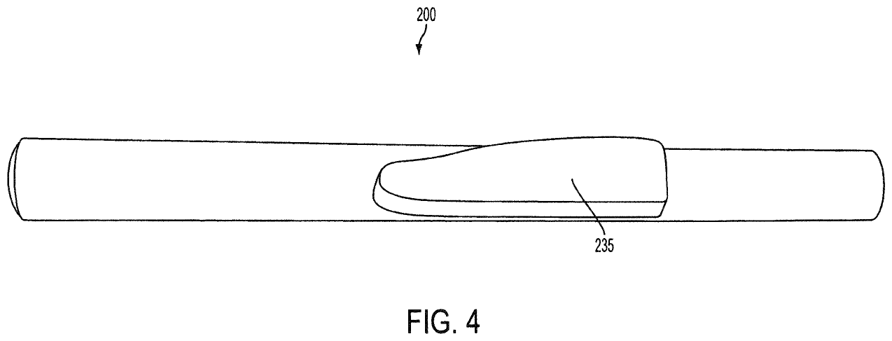

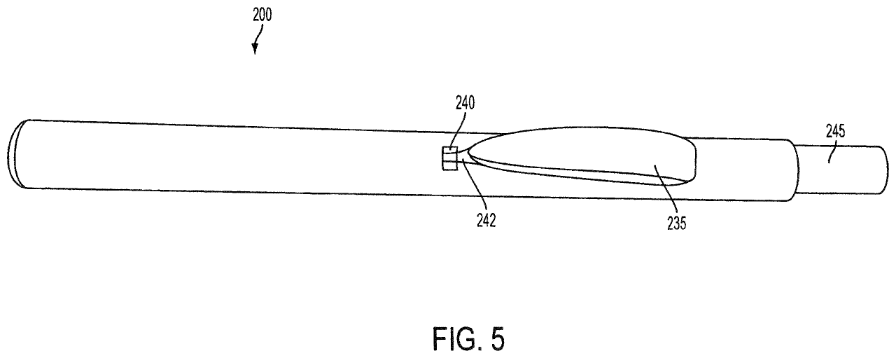

Referring now to FIGS. 4 and 5, another feature of the electronic cigarette 200 is illustrated. Extendable tip 245 is retractable into the casing 15. As shown in FIG. 4, slideable clip 235, shaped substantially like a clip on a pen or the like, is slideable toward one end of the electronic cigarette 200. Rod, or connector 242 connects to the clip 235, with one end of the rod 242 slideably positioned in casing aperture 240.

As the clip 235 is moved toward the end of the electronic cigarette 200, extendable tip 245 extends from the casing 15. The extendable tip 245 may comprise a generally hollows member that a user would grasp with their lips to inhale the vapors exiting through end-cap aperture 82. The extendable tip 245 can be kept clean, and away from contaminants by being kept retracted within the casing 15 when the electronic cigarette 200 is not in use.

Also, the clip 235 may also be used to attach, or secure the electronic cigarette 200 to clothing, purses, pockets or other items. And, in one embodiment, the clip 235 may be shaped to act as a stand for the electronic cigarette 200. The “stand” may prevent direct contact of the extendable tip 245 to an unclean surface when the electronic cigarette 200 is placed on a surface.

An alternative embodiment extendable tip may comprise a rotatable piston (not shown) that includes threads that when rotated, the extendable tip rides upwardly or downwardly on the threads. To prevent the rotatable piston from merely turning in response to the motion of a driving member (not shown) so as not to slide, the interior wall may be provided with a guide member (not shown) that extends along at least a portion of the length of the rotatable piston and mates with a corresponding feature in the casing 15. In a preferred embodiment, the rotatable piston and the interior cross section may be given a shape that is other than circular. Preferably, the rotatable piston may have an oval or ellipsoid shape. In some embodiments, with an integrated reservoir the bulb 230 is T-shaped, L-shaped, or the like.

### Removable Hygienic Wrapper Feature

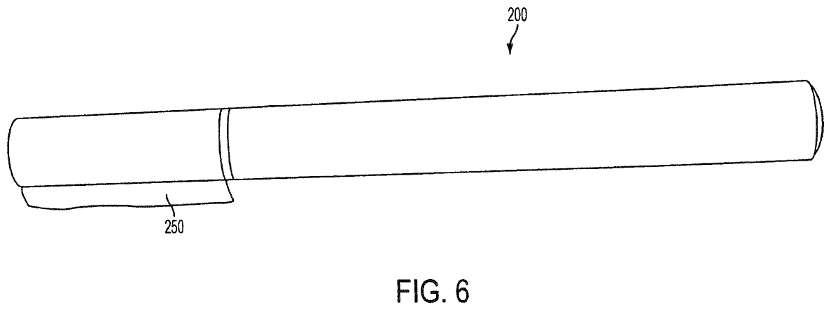

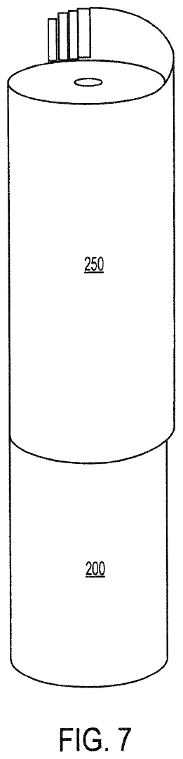

Referring now to FIGS. 6 and 7, another feature of the electronic cigarette 200 is illustrated. Removable wrapper 250 may be located at one distal end of the electronic cigarette 200, where a user would place their lips when drawing on the electronic cigarette 200. For example, in one embodiment, one or more sanitary peel-away wrappers 250 that can be removed as desired are coupled to the electronic cigarette 200, so that a user's lips are not exposed to a dirty or contaminated tip.

### Vapor and Flavor Filter Feature

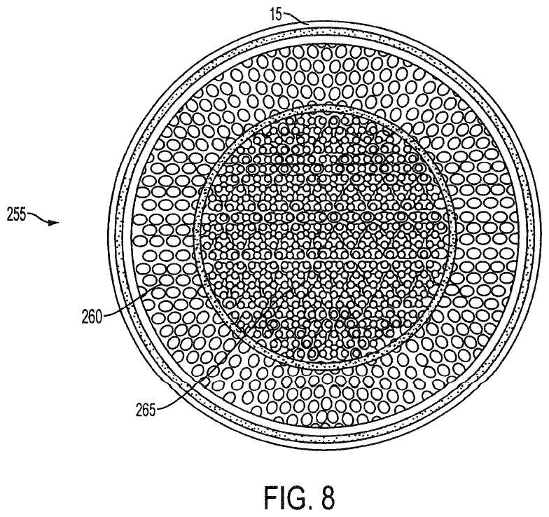

Referring now to FIG. 8, another feature of the electronic cigarette 200 is illustrated. Some embodiments of the electronic cigarette 200 may include a filter assembly 255. The filter assembly 255 may be located at one distal end of the electronic cigarette 200, where a user would place their lips when drawing on the electronic cigarette 200. In one embodiment, the filter assembly 255 includes an inner element 265 and an outer element 260. One or both of the inner and outer elements 265 and 260 may be constructed of microfibers or other materials made of any polyester, polyamides, bamboo, wood or any cellulose fibers or any polymeric material that is capable of absorbing water or vapor, and that is capable of being impregnated with a flavor or chemical compound.

For example, one or both of the inner and outer elements 265 and 260 may comprise filtration and pass through structures comprise flow passages having a high surface area for contact with vapor passing there through, enabling the vapor to become enriched with nicotine, flavors, and other blends, either alone or in combination. In one arrangement, the outer element 260 may comprise a multiplicity of axially elongated fibrous filtering material members disposed concentrically to reduce the amount of water vapor carried into the lungs when inhaling vapors from the electronic cigarette 200.

In one embodiment, the inner element 265 comprises a section capable of dispensing volatile ingredients including nicotine, tobacco flavor, medications, and other flavoring. Reservoirs formed therein may be circular or noncircular in cross section. Absorbent material may be used to retain more volatiles within the section to better wick the volatile vapors when vapor or air is drawn through the electronic cigarette 200. Drawing vapor or air through the inner element 265 transfers vapors to the user. Loading of any material into the inner or outer elements 265 or 260 may be accomplished via sprayer, direct injection or a gaseous atmosphere conductive to preserving nicotine. The fluid mechanic principles of adhesion, cohesion capillary action and surface tension which can cause a thin film may be used. The basic properties, components and applications may be exchanged between the inner and outer elements 265 and 260.

### Transdermal Sleeve Feature

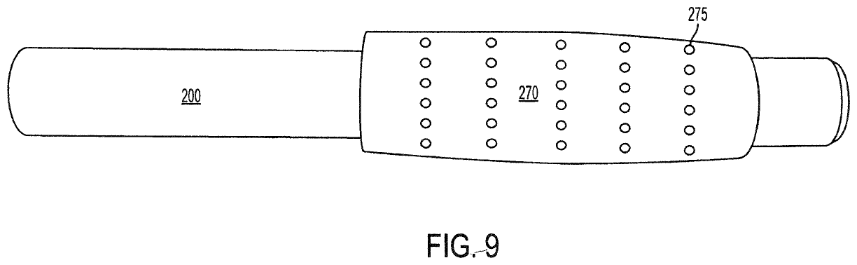

Now referring to FIG. 9, another feature of the electronic cigarette is illustrated. A removable sleeve 270 may be placed over the casing 15 and positioned at any location along the casing 15. The removable sleeve 270 includes a plurality of apertures 275 that may be filled with nicotine or other medications.

In one embodiment, the removable sleeve 270 is constructed of rubber, latex, or a medical grade rubber, latex or plastic. During the manufacturing process, the plurality of apertures, or holes 275 are formed by a mold, or by other means. The plurality of apertures, or holes 275 may then be filled with any desired medication(s) (usually in a gel form) by immersing the removable sleeve 270 in the medication or by injection. When injection is used no holes 275 need be added to the mold. In this embodiment, the removable sleeve 270 is inserted into a device that injects the medications right into the sleeve material. After filling, a cellulite spray is applied to the removable sleeve 270. The cellulite cover retains the medication in the holes 275 until the cellulite is broken when pressure is applied, thereby allowing the medications to provide transdermal drug delivery.

In another embodiment, a transdermal drug delivery system may be built into the cigarette itself without the use of the removable sleeve 270. In this embodiment, an absorbent paper featuring a mesh overlay (not shown) may be employed. When manufactured, a paper is soaked with the desired medication. Then a mesh overly is applied which is made of medical grade rubber, latex or other material as described above in connection with the removable sleeve 270. Next, the paper and mesh (not shown) are covered in a cellulite to seal the medication in. Then, the paper and mesh are wrapped around the desired product, which could be the electronic cigarette 200, or a cardboard tube that looks like a cigarette. When pressure is applied to the paper the pressure removes the cellulite wrapper allowing finger tips to squeeze down the mesh and allow the medicated absorbent paper to provide a transdermal drug delivery

It is appreciated and understood that the term fluid as used herein is exemplary of an inhalation material that can be used with the devices described herein. In additional to liquids, the inhalation material may comprise concentrates, waxes, solid materials, herbs, tobacco. and the like.

It is to be noticed that the term “comprising,” used in the claims, should not be interpreted as being limitative to the means listed thereafter. Thus, the scope of the expression “a device comprising means A and B” should not be limited to devices consisting only of components A and B. It means that with respect to the present invention, the only relevant components of the device are A and B. Put differently, the terms “including”, “comprising” and variations thereof mean “including but not limited to”, unless expressly specified otherwise.

Similarly, it is to be noticed that the term “coupled”, also used in the claims, should not be interpreted as being limitative to direct connections only. Thus, the scope of the expression “a device A coupled to a device B” should not be limited to devices or systems wherein an output of device A is directly connected to an input of device B. It means that there exists a path between an output of A and an input of B which may be a path including other devices or means.

The enumerated listing of items does not imply that any or all of the items are mutually exclusive, unless expressly specified otherwise. The terms “a”, “an” and “the” mean “one or more”, unless expressly specified otherwise.

Elements of the invention that are in communication with each other need not be in continuous communication with each other, unless expressly specified otherwise. In addition, elements of the invention that are in communication with each other may communicate directly or indirectly through one or more other elements or other intermediaries.

One skilled in the art will appreciate that the present invention can be practiced by other than the above-described embodiments, which are presented in this description for purposes of illustration and not of limitation. The specification and drawings are not intended to limit the exclusionary scope of this patent document. It is noted that various equivalents for the particular embodiments discussed in this description may practice the invention as well. That is, while the present invention has been described in conjunction with specific embodiments, itis evident that any alternatives, modifications, permutations and variations will become apparent to those of ordinary skill in the art in light of the foregoing description. Accordingly, it is intended that the present invention embrace all such alternatives, modifications and variations as fall within the scope of the appended claims. The fact that a product, process or method exhibits differences from one or more of the above-described exemplary embodiments does not mean that the product or process is outside the scope (literal scope and/or other legally-recognized scope) of the following claims.

What is claimed is:

1. In an electronic smoking apparatus, a heating device, comprising:
a heating element disposed and encased inside a bulb, wherein a portion of the heating element is exposed exterior of the bulb, the portion delimiting at least one electrical contact; and
the bulb including a reservoir configured to receive and retain an inhalant material; and
wherein the heating device is in electrical communication with a battery of the electronic smoking device and wherein the heating device is in fluid communication with an inhalation material of the electronic smoking device.

2. The heating device of claim 1, wherein the reservoir comprises an indentation in the surface of the bulb.

3. The heating device of claim 1, wherein the reservoir comprises a protrusion extending from the surface of the bulb.

4. The heating device of claim 1, wherein the bulb comprises glass, quartz, or combinations thereof.

5. The heating device of claim 1, wherein the heating element comprises a wire.

6. The heating device of claim 1, wherein the heating element comprises a coil.

7. The heating device of claim 1, wherein the heating element comprises a ribbon.

8. The heating device of claim 1, wherein the bulb comprises beveled ends.

9. The heating device of claim 1, wherein the heating element extends from both ends of the bulb, thereby delimiting an anode and a cathode.

10. The heating device of claim 1, further comprises a sealing element sealing the heating element inside the bulb.

11. The heating device of claim 1, wherein the electrical contact is flush with an end of the bulb.

12. The heating device of claim 11, wherein a magnetic connector is disposed about the electrical contact.

13. In an electronic smoking device, a heating device, comprising an elongated bulb configured to receive and retain therein a heating element wherein the heating device is in electrical communication with a battery of the electronic smoking device and wherein the heating device is in fluid communication with an inhalation material of the electronic smoking device.

14. The heating device of claim 13, including a through-bore disposed lengthwise through the bulb, the through-bore configured to receive and retain therein the heating element.

15. The heating device of claim 13, wherein the bulb includes an indentation, the indentation comprising a reservoir to receive and retain inhalant material.

16. The heating device of claim 13, wherein the bulb includes a protrusion, the protrusion comprising a reservoir to receive and retain inhalant material.

17. The heating device of claim 13, wherein the bulb is substantially cylindrical in shape.

18. The heating device of claim 13, wherein the bulb comprises glass, quartz, or combinations thereof.
